SEQUENCE LISTING (38 sequences)

SEQ ID NO: 1 - protein, 242 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 2 - protein, 10 aa, synthetic construct
GGGSSGGGSG

SEQ ID NO: 3 - protein, 12 aa, synthetic construct
ESKYGPPCPPCP

SEQ ID NO: 4 - protein, 12 aa, synthetic construct
ESKYGPPCPSCP

SEQ ID NO: 5 - protein, 22 aa, synthetic construct
ESKYGPPCPPCPGGGSSGGGSG

SEQ ID NO: 6 - protein, 39 aa, synthetic construct
IEVMYPPPYLDNEKSNGTIIHVKGKHLCPSPLFPGPSKP

SEQ ID NO: 7 - protein, 48 aa, synthetic construct
AKPTTTPAPRPPTPAPTIASQPLSLRPEACRPAAGGAVHTRGLDFACD

SEQ ID NO: 8 - protein, 45 aa, synthetic construct
TTTPAPRPPTPAPTIASQPLSLRPEACRPAAGGAVHTRGLDFACD

SEQ ID NO: 9 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, synthetic construct
ESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, synthetic construct
LCYLLDGILFIYGVILTALFL

SEQ ID NO: 14 - protein, 27 aa, synthetic construct
FWVLVVVGGVLACYSLLVTVAFIIFWV

SEQ ID NO: 15 - protein, 28 aa, synthetic construct
MFWVLVVVGGVLACYSLLVTVAFIIFWV

SEQ ID NO: 16 - protein, 22 aa, synthetic construct
MALIVLGGVAGLLLFIGLGIFF

SEQ ID NO: 17 - protein, 21 aa, synthetic construct
IYIWAPLAGTCGVLLLSLVIT

SEQ ID NO: 18 - protein, 23 aa, synthetic construct
IYIWAPLAGTCGVLLLSLVITLY

SEQ ID NO: 19 - protein, 24 aa, synthetic construct
IYIWAPLAGTCGVLLLSLVITLYC

SEQ ID NO: 20 - protein, 27 aa, synthetic construct
IISFFLALTSTALLFLLFFLTLRFSVV

SEQ ID NO: 21 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, synthetic construct
RSKRSRLLHSDYMNMTPRRPGPTRKHYQPYAPPRDFAAYRS

SEQ ID NO: 23 - protein, 41 aa, synthetic construct
RSKRSRGGHSDYMNMTPRRPGPTRKHYQPYAPPRDFAAYRS

SEQ ID NO: 24 - protein, 42 aa, synthetic construct
KRGRKKLLYIFKQPFMRPVQTTQEEDGCSCRFPEEEEGGCEL

SEQ ID NO: 25 - protein, 42 aa, synthetic construct
ALYLLRRDQRLPPDAHKPPGGGSFRTPIQEEQADAHSTLAKI

SEQ ID NO: 26 - protein, 323 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 27 - protein, 24 aa, synthetic construct
LEGGGEGRGSLLTCGDVEENPGPR

SEQ ID NO: 28 - protein, 354 aa, synthetic construct
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - skipped

SEQ ID NO: 30 - protein, 468 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 650 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 33 - protein, 112 aa, synthetic construct
DIVMTQSPDSLAVSLGERATMSCKSSQSILYSSNQKNYLAWYQQKPGQSPKLLIYWASTRESGVPDRFSGSGSGTDFTLTISSLQAEDVAVYYCHQYLSSYTFGQGTKLEIK

SEQ ID NO: 34 - protein, 22 aa, synthetic construct
MLLLVTSLLLCELPHPAFLLIP

SEQ ID NO: 35 - DNA, 2616 bp, synthetic construct
  source 1..2616: mol_type = other DNA
atgctgctccttgtcacatccctgctgctgtgcgaactgccacatcccgccttcctgctgatcccccaagtgcagctcgtgcagtccggagccgaagtcaagaagcctggcagctccgtcaaggtgtcctgcaaagcctccggctacacctttaccgactacaacatgcactgggtccgccaagcacctggacagggactggagtggattgggtacatctacccttacaacggaggcaccgggtacaaccagaagttcaagtcgaaggccaccattaccgcggacgaatccaccaacaccgcgtatatggagctctcatccttgcggtcggaggacactgccgtgtactactgcgcgaggggtagaccggcaatggactactggggccagggcactctcgtcaccgtgtcctctggtggtggaggctcaggaggagggggatccggtggaggagggagcgatatccagatgacgcagtcaccctcgtccctgagcgcttccgtgggcgatcgcgtgactatcacttgccgggcttccgagtccgtggataactacggaatttcctttatgaactggttccagcaaaagccgggaaaggccccaaagctcctgatctacgccgccagcaatcagggatcgggagtgccctcacggttctccgggagcggttcaggcaccgacttcacccttactatttcgagcctgcaacctgacgatttcgccacttattactgccaacagtccaaggaagtgccgtggacgttcggccaggggaccaaggtggaaatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaagcggggcagaaagaagctgctgtacatcttcaagcagcccttcatgcggcccgtgcagaccacccaggaagaggacggctgcagctgccggttccccgaggaagaggaaggcggctgcgagctgggaggcggcagagtgaagttcagccggtccgccgacgcccctgcctaccagcagggccagaaccagctgtacaacgagctgaacctgggcaggcgggaggaatacgacgtgctggacaagcggagaggccgggaccctgagatgggcggcaagcccaggcggaagaaccctcaggaaggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 36 - protein, 871 aa, synthetic construct
MLLLVTSLLLCELPHPAFLLIPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 37 - DNA, 3162 bp, synthetic construct
  source 1..3162: mol_type = other DNA
atgctgctccttgtcacatccctgctgctgtgcgaactgccacatcccgccttcctgctgatcccccaagtgcagctcgtgcagtccggagccgaagtcaagaagcctggcagctccgtcaaggtgtcctgcaaagcctccggctacacctttaccgactacaacatgcactgggtccgccaagcacctggacagggactggagtggattgggtacatctacccttacaacggaggcaccgggtacaaccagaagttcaagtcgaaggccaccattaccgcggacgaatccaccaacaccgcgtatatggagctctcatccttgcggtcggaggacactgccgtgtactactgcgcgaggggtagaccggcaatggactactggggccagggcactctcgtcaccgtgtcctctggtggtggaggctcaggaggagggggatccggtggaggagggagcgatatccagatgacgcagtcaccctcgtccctgagcgcttccgtgggcgatcgcgtgactatcacttgccgggcttccgagtccgtggataactacggaatttcctttatgaactggttccagcaaaagccgggaaaggccccaaagctcctgatctacgccgccagcaatcagggatcgggagtgccctcacggttctccgggagcggttcaggcaccgacttcacccttactatttcgagcctgcaacctgacgatttcgccacttattactgccaacagtccaaggaagtgccgtggacgttcggccaggggaccaaggtggaaatcaaggagagcaaatacggaccgccgtgtccaccctgtcctgcacccgagttcgaaggcggcccttccgtgttcctgttcccgccgaagcccaaggacaccctgatgatctcgagaaccccggaggtgacctgcgtggtggtggacgtgtcccaggaagatcccgaggtccagttcaattggtacgtggacggcgtggaagtgcacaatgccaagaccaagcccagagaggaacagttccaaagcacctaccgggtggtgtccgtgctgaccgtgctgcaccaggactggctgaacggcaaagagtacaagtgcaaggtgtccaacaagggcctgcccagcagcatcgagaaaaccatcagcaaggccaagggccagccccgcgagccccaggtgtacacactgccccccagccaggaagagatgaccaagaaccaggtgtccctgacctgcctggtcaagggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtctctgagcctgagcctgggcaagatggccctgatcgtgctgggcggagtggccggactgctgctgtttatcggcctgggcatcttcttcaagcggggcagaaagaagctgctgtacatcttcaagcagcccttcatgcggcccgtgcagaccacccaggaagaggacggctgcagctgccggttccccgaggaagaggaaggcggctgcgagctgggaggcggcagagtgaagttcagccggtccgccgacgcccctgcctaccagcagggccagaaccagctgtacaacgagctgaacctgggcaggcgggaggaatacgacgtgctggacaagcggagaggccgggaccctgagatgggcggcaagcccaggcggaagaaccctcaggaaggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 38 - protein, 1053 aa, synthetic construct
MLLLVTSLLLCELPHPAFLLIPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

(19) United States
(12) Patent Application Publication
(10) Pub. No.: US 2026/0250691 A1
(43) Pub. Date: Aug. 27, 2026

(54) CD33 TARGETED CHIMERIC ANTIGEN RECEPTOR MODIFIED T CELLS FOR TREATMENT OF CD33 POSITIVE MALIGNANCIES

(71) Applicant: City of Hope, Duarte, CA (US)

(72) Inventors: Tongyuan Xue, Duarte, CA (US); David A. Horne, Duarte, CA (US); Lihua Elizabeth Budde, Duarte, CA (US); Stephen Forman, Duarte, CA (US); Marissa M. Del Real, Duarte, CA (US)

(21) Appl. No.: 19/457,368

(22) Filed: Jan. 23, 2026

Related U.S. Application Data
(63) Continuation of application No. 17/614,233, filed on Jun. 1, 2020, now Pat. No. 12,565,658.
(60) Provisional application No. 62/855,906, filed on May 31, 2019.

(51) Int. Cl. C12N 15/62; C07K 14/705

(52) U.S. Cl. CPC C12N 15/62; C07K 14/7051

(57) ABSTRACT

Chimeric antigen receptors targeted to CD33 are described together with their use in treating various cancers and in reducing myeloid derived suppressor cells in a patient.

### CLAIM OF PRIORITY

This application is a continuation of U.S. application Ser. No. 17/614,233, filed on Nov. 24, 2021, which is the U.S. National Phase application under 35 U.S.C. § 371 of International Application No. PCT/US2020/035579, filed on Jun. 1, 2020, which claims the benefit of U.S. Provisional Application No. 62/855,906, filed on May 31, 2019. The entire contents of the foregoing are incorporated herein by reference.

### SEQUENCE LISTING

This application contains a Sequence Listing that has been submitted electronically as an XML file named “40056-0052002.XML.” The XML file, created on Jan. 22, 2026, is 44,336 bytes in size. The material in the XML file is hereby incorporated by reference in its entirety.

### TECHNICAL FIELD

This disclosure concerns leukemia-associated CD33-specific chimeric antigen receptor (CAR)-engineered T cells, methods of formulating, and methods of use as anti-cancer agents selective against CD33-positive cells.

### BACKGROUND

Acute myeloid leukemia (AML) is the most common acute leukemia in adults and has the highest mortality rate (Budde et al. (2017) Blood 130:811; Döhner et al. (2015) N Engl J Med 373:1136-52; Schuster et al. (2017) N Engl J Med 377:2545-54). CD33 is expressed on myeloid blasts in 87-98% of AML cases (Ehninger et al. (2014) Blood Cancer 4: e218; Andrews R G et al. (1989) J Exp Med 169:1721-31). CD33 is also expressed on myeloid-dervied suppressor cells (MDSCs), leukemic stem cells (LSCs), and hematopoietic stem cells (HSCs) (Elliot L A et al. (2017) Front Immunol 8:86; Walter R B et al. (2012) Blood 119:6198-208). The current cure rate for primary AML is 35% and decreases with age; thus, there is an urgent unmet need for novel therapies for AML patients, particularly relapsed or refractory (R/R) AML.

### SUMMARY

Described herein are methods for using CD33 targeted CAR T cells (also herein called CD33 CAR T cells) to treat a variety of cancers, for example, acute myeloid leukemia (AML).

Described herein is a nucleic acid molecule encoding a polypeptide comprising a chimeric antigen receptor (CAR), wherein the CAR comprises an amino acid sequence that is at least 85%, 90%, 95%, 96%, 97%, 98%, 99%, or 100% identical SEQ ID NO: 30 or at least about 85%, 90%, 95%, 96%, 97%, 98%, 99%, or 100% identical to SEQ ID NO: 31. Also described is a nucleic acid molecule encoding a polypeptide comprising a chimeric antigen receptor (CAR), wherein the CAR comprises the amino acid sequence of SEQ ID NO: 30 or SEQ ID NO: 31 with no more than 5 (e.g., 1, 2, 3, 4 or 5) single amino acid changes. In certain embodiments the amino acid changes are entirely within the sequence

(SEQ ID NO: 1)
QVQLVQSGAEVKKPGSSVKVSCKASGYTFTDYNMHWVRQAPGQGLEWIGY
IYPYNGGTGYNQKFKSKATITADESTNTAYMELSSLRSEDTAVYYCARGR
PAMDYWGQGTLVTVSSGGGGSGGGGSGGGGSDIQMTQSPSSLSASVGDRV
TITCRASESVDNYGISFMNWFQQKPGKAPKLLIYAASNQGSGVPSRFSGS
GSGTDFTLTISSLQPDDFATYYCQQSKEVPWTFGQGTKVEIK
or the sequence
(SEQ ID NO: 32)
QVQLVQSGAEVKKPGSSVKVSCKASGYTFTDYNMHWVRQAPGQGLEWIGY
IYPYNGGTGYNQKFKSKATITADESTNTAYMELSSLRSEDTAVYYCARGR
PAMDYWGQGTLVTVSS
or the sequence
(SEQ ID NO: 33)
DIVMTQSPDSLAVSLGERATMSCKSSQSILYSSNQKNYLAWYQQKPGQSP
KLLIYWASTRESGVPDRFSGSGSGTDFTLTISSLQAEDVAVYYCHQYLSS
YTFGQGTKLEIK.

Also described is an expression vector comprising the nucleic acid molecule (e.g., a viral vector or a lentiviral vector). Also described is a population of human T cells or NK cells transduced by a vector or comprising the nucleic acid molecule described above. In various embodiments the cells comprise or consist of or consist essentially of: central memory T cells, naive memory T cells, pan T cells, NK cells, or PBMC substantially depleted for CD25+ cells and CD14+ cells.

Also described is method of treating a leukemia or a cancer in a patient comprising administering a population of autologous or allogeneic human T cells or NK transduced by a vector comprising the nucleic acid molecule described above or T cells or NK cells comprising a nucleic acid molecule described herein, wherein the leukemia comprises cells expressing CD33. In various embodiments: the chimeric antigen receptor is administered locally or systemically; the CD33-expressing cells are cancerous T cells or T-regulatory cells; the chimeric antigen receptor is administered by single or repeat dosing; the patient is administered a hypomethylating agent prior to or in conjunction the population of autologous or allogeneic human T cells; the hypomethylating agent is a DNA methyltransferase inhibitor; the hypomethylating agent is selected from 5-azacytidine and decitabine; the cancer is a hematologic cancer selected from the group acute myeloid leukemia (AML); myelodysplastic syndrome; myeloproliferative neoplasms; chronic myeloid leukemia (CML); and blastic plasmacytoid dendritic cell neoplasm.

Also described is method for reducing myeloid-derived suppressor cells (MDSC) in a patient suffering from cancer (e.g., a solid tumor) the method comprising administering immune cells comprising a nucleic acid molecule described herein to the patient. In some cases, the MDSC is lineage negative (LIN−), HLA-DR negative, and CD33 positive. In some case, a CD33 CAR-expressing cell described herein targets a MDS blast and a MDSC. In embodiments, a CD33 CAR-expressing cell described herein is used to treat multiple myeloma, chronic lymphocytic leukemia (CLL), or solid malignancies such as ovarian cancer, colon cancer, or breast cancer.

Also described is a method of preparing CD33 CAR T cells comprising: providing a population of autologous or allogeneic human T cells or NK and transducing the T cells or NK cells

Described herein is a nucleic acid molecule comprising a nucleotide sequence encoding a chimeric antigen receptor (CAR), wherein the chimeric antigen receptor comprises: an scFv targeting CD33, a spacer, a transmembrane domain, a 41-BB co-stimulatory domain, and a CD3 ¿ signaling domain.

In various embodiments: the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; the spacer comprises 20-150 amino acids and is located between the scFv and the transmembrane domain; the transmembrane domain is a CD4 transmembrane domain or variant thereof having 1-5 amino acid modifications; the transmembrane domain is a CD4 transmembrane domain; the chimeric antigen receptor comprises a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications; the spacer region comprises an amino acid sequence selected from the group consisting of SEQ ID NOs: 2-12 or a variant thereof having 1-5 amino acid modifications; the spacer comprises an IgG hinge region; the spacer comprises 10-50 amino acids; the 4-1BB costimulatory domain comprises the amino acid sequence of SEQ ID NO: 24 or a variant thereof having 1-5 amino acid modifications; the CD3ζ signaling domain comprises the amino acid sequence of SEQ ID NO:21; a linker of 3 to 15 amino acids is located between the 4-1BB costimulatory domain and the CD3 ζ signaling domain or variant thereof; the CAR comprises the amino acid sequence of SEQ ID NO: 30 or 31 or a variant thereof having 1-5 amino acid modifications, e.g., 1-5 single amino acid substitutions; the scFv comprises the amino acid sequence of SEQ ID NO:1; the nucleic acid molecule of claim 1.

Also disclosed herein is: a viral vector comprising a nucleic acid molecule described herein; a population of human T cells (e.g., a population comprising central memory T cells) transduced by a vector comprising a nucleic acid molecule described herein.

Also described herein is a method of treating CD33 positive cancers (including, e.g., AML, R/R AML, acute lymphoblastic leukemia (ALL), myelodysplastic syndromes (MDS), myeloma, myeloproliferative neoplasms, other CD33⁺ hematologic malignancies, and the like) in a patient comprising administering a population of autologous or allogeneic human T cells transduced by a vector comprising a nucleic acid molecule described herein, wherein the cancer (or disease or disorder) comprises cells expressing CD33. In various embodiments: the chimeric antigen receptor is administered locally or systemically; the CD33-expressing cells are cancerous T cells; and the chimeric antigen receptor is administered by single or repeat dosing.

In various embodiments: the chimeric antigen receptor comprises: a CD33 scFv (e.g., an scFv comprising the amino acid sequence

QVQLVQSGAEVKKPGSSVKVSCKASGYTFTDYNMHWVRQAPGQGLEWIGYIYPYNGG TGYNQKFKSKATITADESTNTAYMELSSLRSEDTAVYYCARGRPAMDYWGQGTL VTVS SGGGGSGGGGSGGGGSDIQMTQSPSSLSASVGDRVTITCRASESVDNYGISFMNWFQQK PGKAPKLLIYAASNQGSGVPSRFSGSGSGTDFTLTISSLQPDDFATYYCQQSKEVPWTFG QGTKVEIK (SEQ ID NO:1) with up to 10 single amino acid substitutions.

Also described are T cells harboring a vector expressing the CD33 CAR. In various embodiments: at least 20%, 30%, or 40% of the transduced human T cells are central memory T cells; at least 30% of the transduced human T cells are CD4+ and CD62L+ or CD8+ and CD62L+. In various embodiments: the population of human T cells comprise a vector expressing a chimeric antigen receptor comprising an amino acid sequence selected from SEQ ID NOs: 30 or 31 or a variant thereof having 1-5 amino acid modifications (e.g., 1 or 2) amino acid modifications (e.g., substitutions); the population of human T cells comprises central memory T cells (T_{CM} cells) e.g., at least 20%, 30%, 40%, 50% 60%, 70%, 80% of the cells are T cm cells, or the population of T cells comprises a combination of central memory T cells, naïve T cells and stem central memory cells (T_{CM/SCM/N} cells) e.g., at least 20%, 30%, 40%, 50% 60%, 70%, 80% of the cells are T_{CM/SCM/N} cells. In some embodiments, the population of T cells includes both CD4+ cells and CD8+ cells (e.g., at least 20% of the CD3+ T cells are CD4+ and at least 3% of the CD3+ T cells are CD8+ and at least 70, 80 or 90% are either CD4+ or CD8+; at least 15%, 20%, 25%, 30%, 35%, 40%, 50%, 60% of the cells CD3+ cells are CD4+ and at least 4%, 5%, 8%, 10%, 20 of the CD3+ cells are CD8+ cells). In some embodiments, the population of human T cells are autologous to the patient. In some embodiments, the population of human T cells are allogenic to the patient.

### CD33 Targeted CAR

The CD33 targeted CAR described herein include a CD33 targeting scFv. In some embodiments, an scFv comprising the amino acid sequence:

(SEQ ID NO: 1)
QVQLVQSGAEVKKPGSSVKVSCKASGYTFTDYNMHWVRQAPGQGLEWIGY
IYPYNGGTGYNQKFKSKATITADESTNTAYMELSSLRSEDTAVYYCARGR
PAMDYWGQGTLVTVSSGGGGSGGGGSGGGGSDIQMTQSPSSLSASVGDRV
TITCRASESVDNYGISFMNWFQQKPGKAPKLLIYAASNQGSGVPSRFSGS
GSGTDFTLTISSLQPDDFATYYCQQSKEVPWTFGQGTKVEIK
or comprising the sequence
(SEQ ID NO: 32)
QVQLVQSGAEVKKPGSSVKVSCKASGYTFTDYNMHWVRQAPGQGLEWIGY
IYPYNGGTGYNQKFKSKATITADESTNTAYMELSSLRSEDTAVYYCARGR
PAMDYWGQGTLVTVSS
and the sequence
(SEQ ID NO: 33)
DIVMTQSPDSLAVSLGERATMSCKSSQSILYSSNQKNYLAWYQQKPGQSP
KLLWWASTRESGVPDRFSGSGSGTDFTLTISSLQAEDVAVYYCHQYLSSY
TFGQGTKLEIK
joined by a flexible linker.

A useful CD33 CAR can consist of or comprises the amino acid sequence of SEQ ID NO: 30 or 31 (mature CAR lacking a signal sequence) or the CD33 CAR can consist of or comprise the amino acid sequence of SEQ ID NO: 30 or 31 preceded by a GMCSFRa signal sequence (SEQ ID NO: 34) (immature CAR having a GMCSFRa signal sequence;

(MLLLVTSLLLCELPHPAFLLIP; SEQ ID NO:34). =The CAR can be expressed with additional sequences that are useful for monitoring expression, for example, a T2A skip sequence and a truncated EGFRt. The CAR can be expressed with additional sequences that are useful for monitoring expression, for example, a T2A skip sequence and a truncated CD19t. Thus, the CAR can comprise or consist of the amino acid sequence of SEQ ID Nos: 30, or 31 or can comprise or consist of an amino acid sequence that is at least 95%, 96%, 97%, 98% or 99% identical to SEQ ID Nos: 30, or 31. The CAR can comprise or consist of the amino acid sequence of any of SEQ ID Nos: 30, or 31 with up to 1, 2, 3, 4 or 5 amino acid changes (preferably conservative amino acid changes). The CAR can comprise an scFv targeted to CD33, e.g., an scFv comprising SEQ ID NO: 1 with up to 1, 2, 3, 4 or 5 amino acid changes (preferably conservative amino acid changes or an scFv comprising SEQ ID NO:32 with up to 1, 2, 3, 4 or 5 amino acid changes (preferably conservative amino acid changes) and SEQ ID NO:33 with up to 1, 2, 3, 4 or 5 amino acid changes (preferably conservative amino acid changes) joined by a flexible linker.

In some embodiments, the nucleic acid encoding amino acid sequences SEQ ID NOs: 1-34 are codon optimized. In some embodiments, the nucleic acid encoding amino acid sequences SEQ ID NOs: 1-34 are not codon optimized.

### Spacer Region

The CAR described herein can include a spacer located between the CD33 targeting domain (i.e., a CD33 targeted ScFv or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge (S228P) + | 22 aa | ESKYGPPCPPCPGGGSSGGGSG (SEQ ID NO: 5) |
| linker | | |
| CD28 hinge | 39 aa | IEVMYPPPYLDNEKSNGTIIHVKGKHLCPSPLFPGPSKP |
| | | (SEQ ID NO: 6) |
| CD8 hinge-48aa | 48 aa | AKPTTTPAPRPPTPAPTIASQPLSLRPEACRPAAGGAVHTRGLDFACD |
| | | (SEQ ID NO: 7) |
| CD8 hinge-45aa | 45 aa | TTTPAPRPPTPAPTIASQPLSLRPEACRPAAGGAVHTRGLDFACD |
| | | (SEQ ID NO: 8) |
| IgG4(HL-CH3) | 129 aa | ESKYGPPCPPCPGGGSSGGGSGGQPREPQVYTLPPSQEEMTKNQVSLTCL |
| (includes S228P in | | VKGFYPSDIAVEWESNGQPENNYKTTPPVLDSDGSFFLYSRLTVDKSRWQE |
| hinge) | | GNVFSCSVMHEALHNHYTQKSLSLSLGK (SEQ ID NO: 9) |
| IgG4(L235E, N297Q) | 229 aa | ESKYGPPCPSCPAPEFEGGPSVFLFPPKPKDTLMISRTPEVTCVVVDVSQED |
| | | PEVQFNWYVDGVEVHQAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 10) |
| IgG4(S228P, L235E, | 229 aa | ESKYGPPCPPCPAPEFEGGPSVFLFPPKPKDTLMISRTPEVTCVVVDVSQED |
| N297Q) | | PEVQFNWYVDGVEVHQAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 11) |
| IgG4(CH3) | 107 aa | GQPREPQVYTLPPSQEEMTKNQVSLTCLVKGFYPSDIAVEWESNGQPENN |
| | | YKTTPPVLDSDGSFFLYSRLTVDKSRWQEGNVFSCSVMHEALHNHYTQKSL |
| | | SLSLGK (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise a IgG4 hinge region having the sequence ESKYGPPCPSCP (SEQ ID NO:4) or ESKYGPPCPPCP (SEQ ID NO:3). The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIA VEWESNGQPENNYKTTPPVLD SDGSFFLYSRLTVDKSRWQEGNVFSCSVMHEALHNHYTQKSLSLSLGK (SEQ ID NO: 12). Thus, the entire linker/spacer region can comprise the sequence: ESKYGPPCPPCPGGGSSGGGSGGQPREPQVYTLPPSQEEMTKNQVSLTCLVKGFYPSDIA VEWESNGQPENNYKTTPPVLDSDGSFFLYSRLTVDKSRWQEGNVFSCSVMHEALHNHY TQKSLSLSLGK (SEQ ID NO:11). In some cases, the spacer has 1, 2, 3, 4, or 5 single amino acid changes (e.g., conservative changes) compared to SEQ ID NO:11. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the. Table 2 includes examples of suitable transmembrane domains. Where a spacer region is present, the transmembrane domain (TM) is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 13) |
| CD28 | NM_006139 | 27 aa | FWVLVVVGGVLACYSLLVTVAFIIF |
| | | | WV (SEQ ID NO: 14) |
| CD28(M) | NM_006139 | 28 aa | MFWVLVVVGGVLACYSLLVTVAFII |
| | | | FWV (SEQ ID NO: 15) |
| CD4 | M35160 | 22 aa | MALIVLGGVAGLLLFIGLGIFF |
| | | | (SEQ ID NO: 16) |
| CD8tm | NM_001768 | 21 aa | IYIWAPLAGTCGVLLLSLVIT |
| | | | (SEQ ID NO: 17) |
| CD8tm2 | NM_001768 | 23 aa | IYIWAPLAGTCGVLLLSLVITLY |
| | | | (SEQ ID NO: 18) |
| CD8tm3 | NM_001768 | 24 aa | IYIWAPLAGTCGVLLLSLVITLYC |
| | | | (SEQ ID NO: 19) |
| 41BB | NM_001561 | 27 aa | IISFFLALTSTALLFLLFFLTLRF |
| | | | SVV (SEQ ID NO: 20) |

### Costimulatory Domain

The costimulatory domain can be any domain that is suitable for use with a CD3ζ signaling domain. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 of 5 amino acid changes (preferably conservative) compared to SEQ ID NO:24.

The costimulatory domain(s) are located between the transmembrane domain and the CD35 signaling domain. Table 3 includes examples of suitable costimulatory domains together with the sequence of the CD32 signaling domain.

TABLE 3
CD3ζ Domain and Examples of Costimulatory
Domains

| Name | Accession | Length | Sequence |
|---|---|---|---|
| CD3ζ | J04132.1 | 113 aa | RVKFSRSADAPAYQQGQNQLYNEL |
| | | | NLGRREEYDVLDKRRGRDPEMGGK |
| | | | PRRKNPQEGLYNELQKDKMAEAYS |
| | | | EIGMKGERRRGKGHDGLYQGLSTA |
| | | | TKDTYDALHMQALPPR |
| | | | (SEQ ID NO: 21) |
| CD28 | NM_006139 | 42 aa | RSKRSRLLHSDYMNMTPRRPGPTR |
| | | | KHYQPYAPPRDFAAYRS |
| | | | (SEQ ID NO: 22) |
| CD28gg* | NM_006139 | 42 aa | RSKRSRGGHSDYMNMTPRRPGPTR |
| | | | KHYQPYAPPRDFAAYRS |
| | | | (SEQ ID NO: 23) |
| 41BB | NM_001561 | 42 aa | KRGRKKLLYIFKQPFMRPVQTTQE |
| | | | EDGCSCRFPEEEEGGCEL |
| | | | (SEQ ID NO: 24) |
| OX40 | | 42 aa | ALYLLRRDQRLPPDAHKPPGGGSF |
| | | | RTPIQEEQADAHSTLAKI |
| | | | (SEQ ID NO: 25) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD35 signaling domain and a short linker consisting of 2-10, e.g., 3 amino acids (e.g., GGG) is can be positioned between the costimulatory domain and the CD33 signaling domain.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to:

RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO:21). In some cases, the CD32 signaling has 1, 2, 3, 4 of 5 amino acid changes (preferably conservative) compared to SEQ ID NO:21.

### Truncated EGFR and Truncated CD19

The CD3ζ signaling domain can be followed by a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to: LVTSLLLCELPHP AFLLIPRKVCNGIGIGEFKDSLSINATNIKHFKNCTSISGDLHILPVAFR GDSFTHTPPLDPQELDILKTVKEITGFLLIQAWPENRTDLHAFENLEIIRGRTKQHGQFSL AVVSLNITSLGLRSLKEISDGDVIISGNKNLCYANTINWKKLFGTSGQKTKIISNRGENSC KATGQVCHALCSPEGCWGPEPRDCVSCRNVSRGRECVDKCNLLEGEPREFVENSECIQC HPECLPQAMNITCTGRGPDNCIQCAHYIDGPHCVKTCPAGVMGENNTLVWKYADAGH VCHLCHPNCTYGCTGPGLEGCPTNGPKIPSIATGMVGALLLLLVVALGIGLFM (SEQ ID NO: 28). In some cases, the truncated EGFR has 1, 2, 3, 4 of 5 amino acid changes (preferably conservative) compared to SEQ ID NO:28.

Alternatively the CD3ζ signaling domain can be followed by a ribosomal skip sequence (e.g., LEGGGEGRGSLLTCGDVEENPGPR; SEQ ID NO:27) and a truncated CD19R having a sequence that is at least 90%, at least 95%, at least 98% identical to or identical to:

(SEQ ID NO: 26)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCVPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKR

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In some cases, the CD33 CAR can be produced using a vector in which the CAR open reading frame is followed by a T2A ribosome skip sequence and a truncated EGFR (EGFRt), which lacks the cytoplasmic signaling tail. In this arrangement, co-expression of EGFRt provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt incorporated in the CD33 CAR lentiviral vector can act as suicide gene to ablate the CAR+ T cells in cases of treatment-related toxicity.

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte, and most preferably an autologous T lymphocyte.

Various T cell subsets isolated from the patient can be transduced with a vector for CAR expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of an CD33 CAR as well as a non-immunogenic surface marker for in vivo detection, ablation, and potential ex vivo selection. The activated/genetically modified CD33 central memory T cells can be expanded in vitro with IL-2/IL-15 and then cryopreserved. Additional methods of preparing CAR T cells can be found in PCT/US2016/043392.

In another aspect, the CD33 CAR expressing cell is an immune effector cell such as T cell or an NK cell that harbor an exogenous RNA molecule, e.g., an in vitro transcribed RNA or synthetic RNA comprising nucleic molecule acid encoding a CAR molecule described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety for any and all purposes. In case of conflict, the present specification, including definitions, will control.

Other features and advantages of the invention will be apparent from the following detailed description and figures, and from the claims.

### DESCRIPTION OF DRAWINGS

FIG. 1 shows a schematic depicting a representative CD33 CAR construct.

FIGS. 2A-2C show results from experiments with CD33 (CD8) CAR T cells where EGFRt is used as a tracking marker and its expression was stable through 13 days culture duration. Mock (untransduced; FIG. 2A) and CD33 CAR T cells (FIG. 2B) were elevated by flow cytometry for EGFR expression to detect expression of CARs. Both mock and CD33 CAR T cells exhibited robust expansion during ex vivo culture (FIG. 2C).

FIG. 3 shows CD33 expression on human AML cell lines. Human Burkitt's lymphoma cell line RAJI was used as a negative control.

FIGS. 4A-4C shows effector functions and killing activities of CD33 (CD8) CAR T cells against CD33-expressing AML cell lines. FIG. 4A shows results from degranulation assay using an E:T ratio of 2:1 of mock or CD33 CAR T cells against AML cells. FIG. 4B shows results from intracellular IFN-γ staining assay using an E:T ratio of 1:1 of mock or CD33 CAR T cells against AML cells. FIG. 4C shows results from rechallenge assay using E:T ratios of 1:5, 1:10, and 1:20 of mock or CD33 CAR T cells against AML cells. RAJI cell line was used as a negative control.

FIG. 5 shows a representative in vivo study using a NOD-SCID-IL2Rg^{null} (NSG) xenograft model with MOLM-14-ffluc cell line, an AML cell line with high CD33 expression levels.

FIGS. 6A-6B show the ability of CD33 (CD8) CAR T cells to kill CD33-expressing tumor lines in vivo. FIG. 6A shows 3×10⁶ CD33 CAR T cells, compared with 3×10⁶ mock T cells or untreated groups, significantly reduced leukemic burden in vivo. FIG. 6B shows 3×10⁶ CD33 CAR T cells, compared with 3×10⁶ mock T cells or untreated groups, significantly prolonged overall survival in vivo.

FIG. 7 shows CD33 expression on AML cell lines in the presence of 100 nM decitabine or DMSO for 2 consecutive days. RAJI cell line was used as a negative control.

FIG. 8 shows a schematic depicting the killing activities of decitabine in combination with CD33 (CD8) CAR T cells in vitro. AML cell lines were treated with 10 nM decitabine or DMSO for 2 consecutive days, followed by CD33 CAR or mock T cells in an E:T ratio of 1:50. Another 48 hours later, the killing activities in each group were determined using flow cytometry. RAJI cell line was used as a negative control.

FIGS. 9A-9B show mean fluorescence intensities (MFI) of programmed death-ligand 1 (PD-L1) and c-type lectin-like molecule-1 (CLL-1) expression on residual AML cells, following decitabine or DMSO in combination with CD33 (CD8) CAR or mock T cells. RAJI cell line was used as a negative control.

FIG. 10 shows a representative in vivo study using a NSG xenograft model with MOLM-14-ffluc that received decitabine in combination with CD33 CAR T cell therapies.

FIGS. 11A-11B show the results of combinational efficacies of decitabine and CD33 (CD8) CAR T cells in vivo. FIG. 11A shows 0.5 mg/kg/d×5 d decitabine, followed by 1×10⁶ CD33 CAR T cells, significantly decreased leukemic burden in vivo. FIG. 11B shows combinational treatment of 0.5 mg/kg/d×5 d decitabine and 1×10⁶ CD33 CAR T cells significantly prolonged overall survival in vivo.

FIG. 12 shows residual AML cells that survived the treatment with 10 nM decitabine and 1:50 E:T ratio of CD33 (CD8) CAR T cells significantly increased PD-L1 expression levels.

FIG. 13 shows anti-PD-1 antibody (10 μg/mL) significantly enhanced the anti-AML efficacy of CD33 (CD8) CAR T cells at an E:T ratio of 1:75.

FIGS. 14A-14B show the annotated amino acid sequences of CD33 CD8 CAR (A; SEQ ID NO: 30) and CD33 EQ CAR (B; SEQ ID NO:31). The alternating underlining indicates the various components.

FIGS. 15A-15F show the annotated nucleic acid and corresponding amino acid sequences of (A-C) CD33 CD8 CAR preceded by a GMCSR signal sequence and followed by a T2A skip sequence and EGFRt (SEQ ID NO: 35 (nucleic acid) and SEQ ID NO: 36 (amino acid)); and (D-F) CD33 EQ CAR preceded by a GMCSR signal sequence and followed by a T2A skip sequence and EGFRt (SEQ ID NO: 35 (nucleic acid) and SEQ ID NO: 36 (amino acid)). The codon optimized portions of each nucleic acid sequence are in bold.

### DETAILED DESCRIPTION

In this disclosure the generation and anti-tumor efficacy of CAR with a humanized anti-human CD33 scFv antigen-binding domain and, in some embodiments, a 4-1BB intracellular co-stimulatory signaling domain are described. The CD33 CAR T cells exhibited potent antigen-dependent cytotoxicity against CD33-expressing human cancer lines. Intraperitoneal or intravenous in vivo delivery of CD33 CAR T cells in human leukemia/lymphoma murine tumor models conferred elimination of antigen-positive disease and extension of overall survival.

### Examples

The invention is further described in the following examples, which do not limit the scope of the invention described in the claims.

### Example 1: Construction of CD33 CAR T Cells Containing Differing Linkers

The studies described below show that CD33 CAR can be stably expressed on primary T cells.

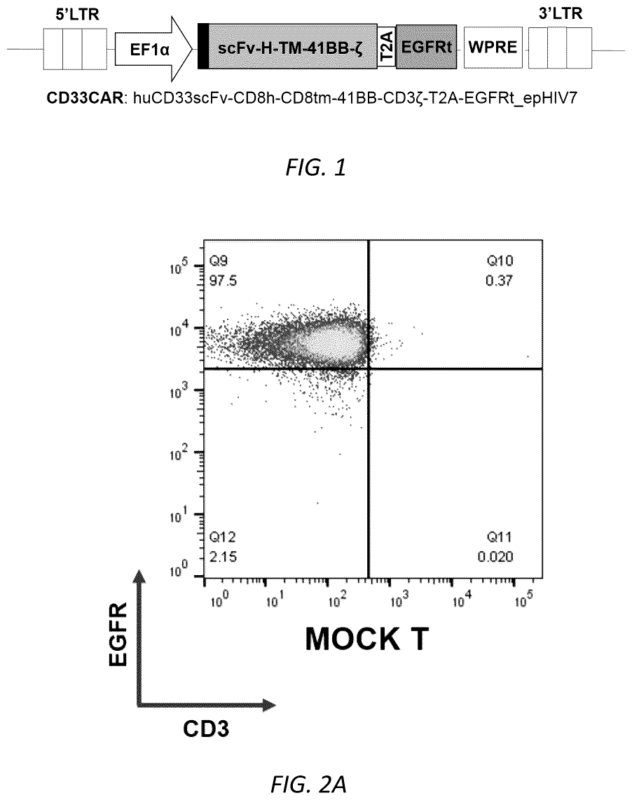

Multiple CD33-targeting CAR constructs were designed. All of the constructs expressed the same codon optimized, humanized CD33 single chain variable fragment. In some embodiments, the CAR constructs also included a CD4 transmembrane domain (TM), a 41BB costimulatory domain, a CD3 zeta domain. In some embodiments, the CAR constructs also included a CD8 transmembrane domain (TM), a 41BB costimulatory domain, a CD3 zeta domain. A representative schematic of a CD33 CAR is shown in FIG. 1. The CARs were co-expressed with truncated EGFR, which served as a marker for the successful transduction of the cells with the CAR construct. In some embodiments, the CD33 CAR constructs differ in their linker and transmembrane domains. Without being bound by theory, differing lengths in the extracellular portion of the construct may provide differences in the CARs ability to bind the antigen and transmit activation signals after antigen binding. These differences could also result differential killing of CD33 expressing tumor cells.

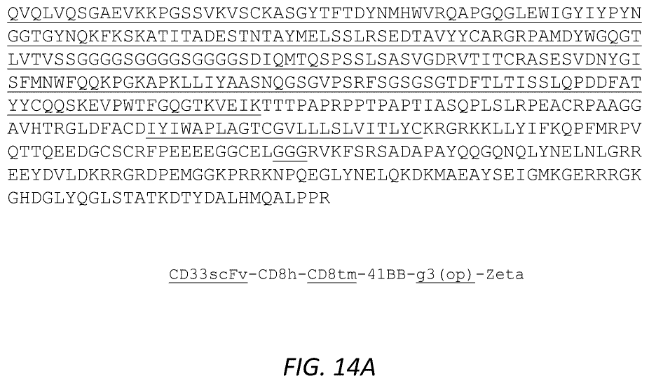

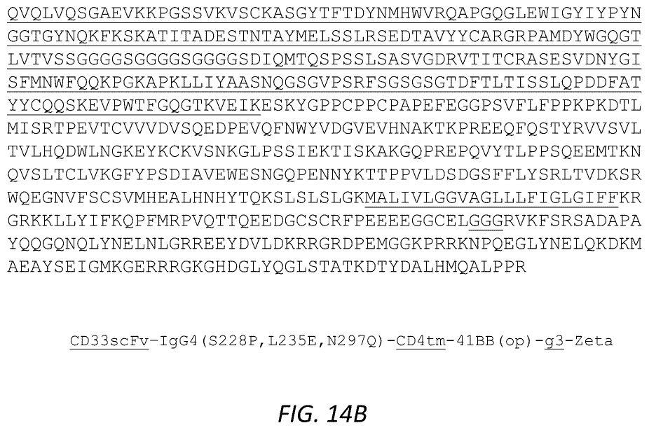

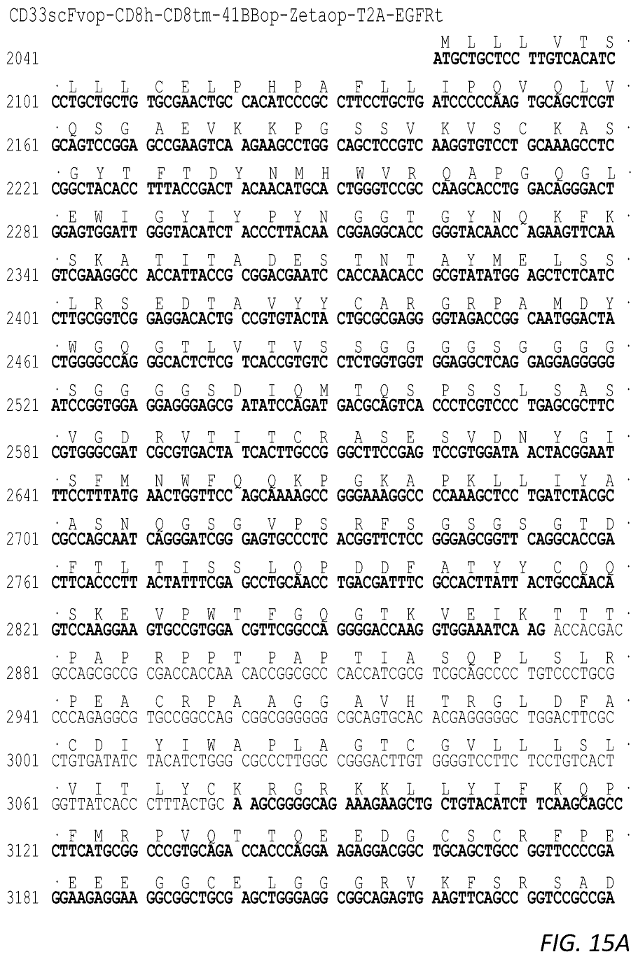

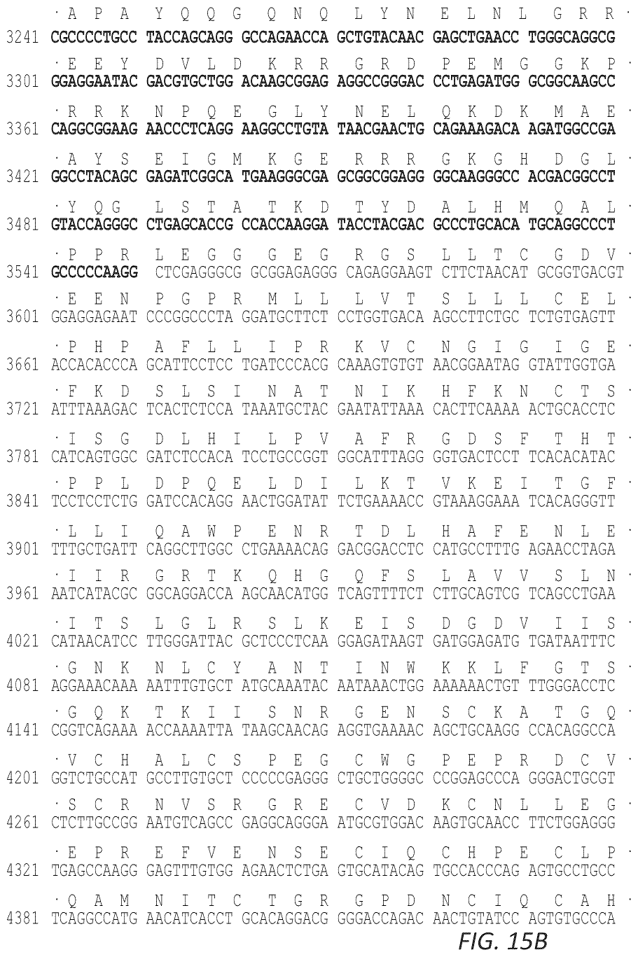

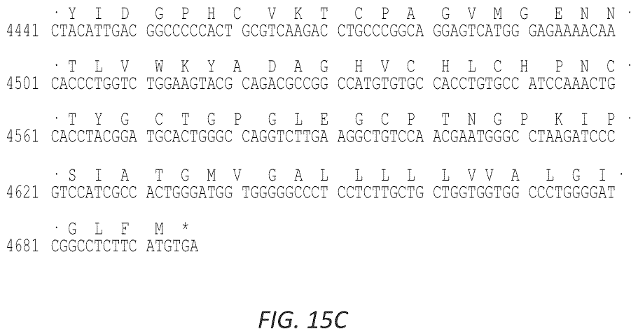

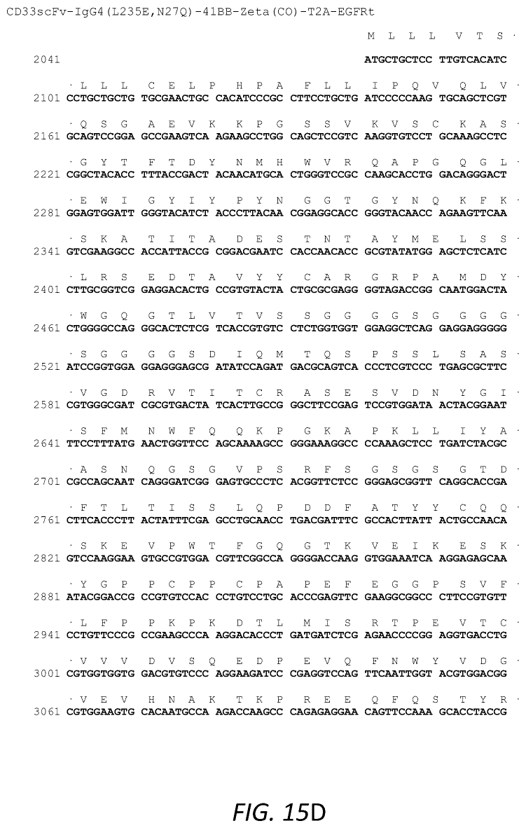

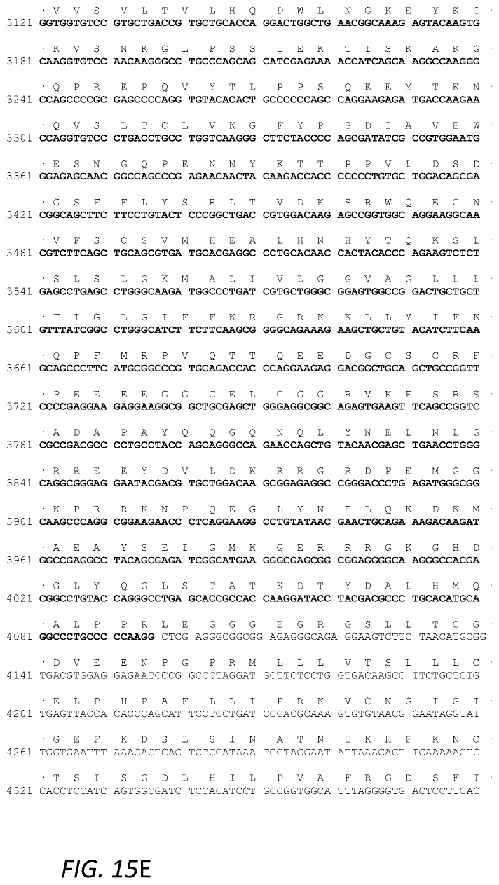

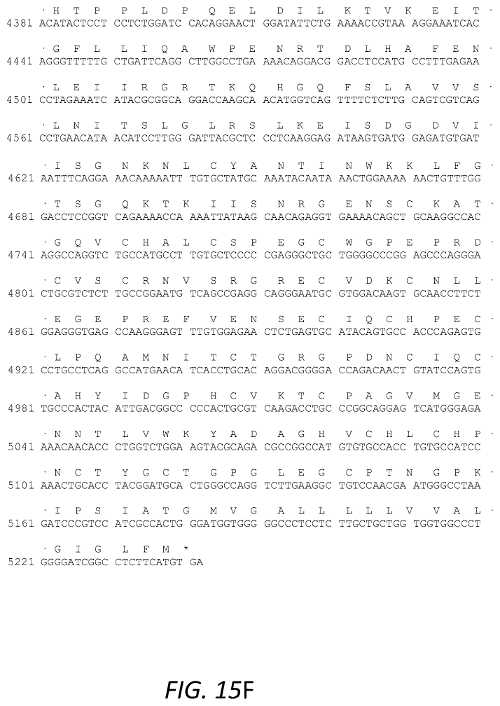

FIG. 14A depicts the amino acid sequence of a CD33 CAR comprising a CD33-targeted scFv, a CD8 hinge (spacer), a CD8 transmembrane domain, a 41-BB co-stimulatory domain joined by a GGG linker to a CD3 zeta stimulatory domain (SEQ ID NO: 30). FIG. 14B depicts the amino acid sequence of a CD33 CAR comprising a CD33-targeted scFv, an IgG4 spacer (IgG4 (S228P, L235E,N297Q)), a CD4 transmembrane domain, a 41-BB co-stimulatory domain joined by a GGG linker to a CD3 zeta stimulatory domain (SEQ ID NO: 31).

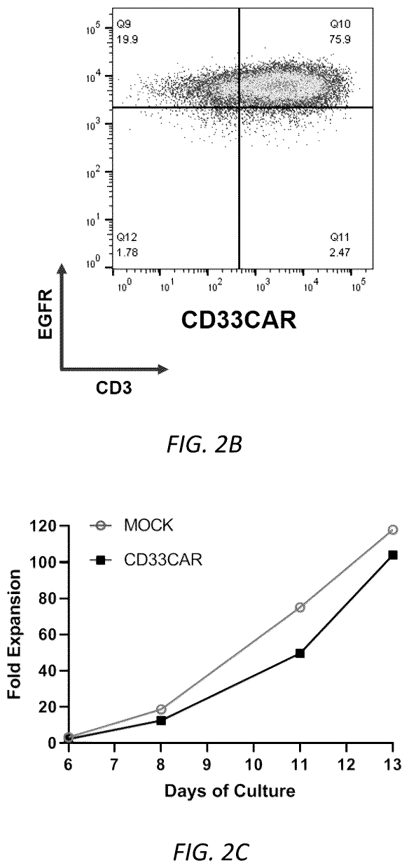

CD33 CAR lentivirus was used to transduce human healthy donor-derived peripheral blood mononuclear cells depleted of CD14+ and CD25+ cells (dPBMC), T (n/mem) cells, Pan T cells, as previously described (Priceman S J, Gerdts E A, Tilakawardane D, Kennewick K T, Murad J P, Park A K, Jeang B, Yamaguchi Y, Yang X, Urak R, Weng L, Chang W C, Wright S, Pal S, Reiter R E, Wu A M, Brown C E, Forman S J. Co-stimulatory signaling determines tumor antigen sensitivity and persistence of CAR T cells targeting PSCA+ metastatic prostate cancer. Oncoimmunology. 2018; 7 (2): e1380764), as well as other cells types such as enriched T-cells (EasySep Human T cell isolation Kit. StemCell Technologies). Using EGFRt as a tracking marker, flow cytometry was used to show CAR expression as described above. A representative CD33 CAR construct was stably expressed in T cells (FIGS. 2A-2C). Seven days after cells were transduced, cells were stained with anti-EGFR to mark successful incorporation of the CD33 CAR construct. The CD33 (CD8) CAR was stably expressed at 15 days post-transduction (FIG. 2C).

### Example 2: CD33 Expression in AML Cell Lines

The studies described below examined CD33 expression on various AML cell lines.

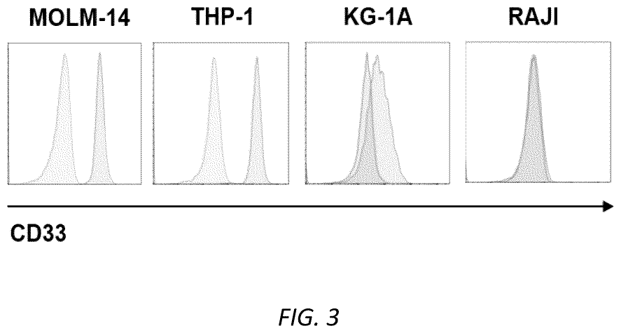

As shown in FIG. 3, CD33 expression was highest in MOLM-14 and THP-1 cell lines. THe KG-1A cell line exhibited intermediate CD33 expression. CD33 expression was lowest in the Raji cell line.

### Example 3: CD33 CAR T Cells Exhibit Potent and Specific Cell Killing and Effector Function In Vitro

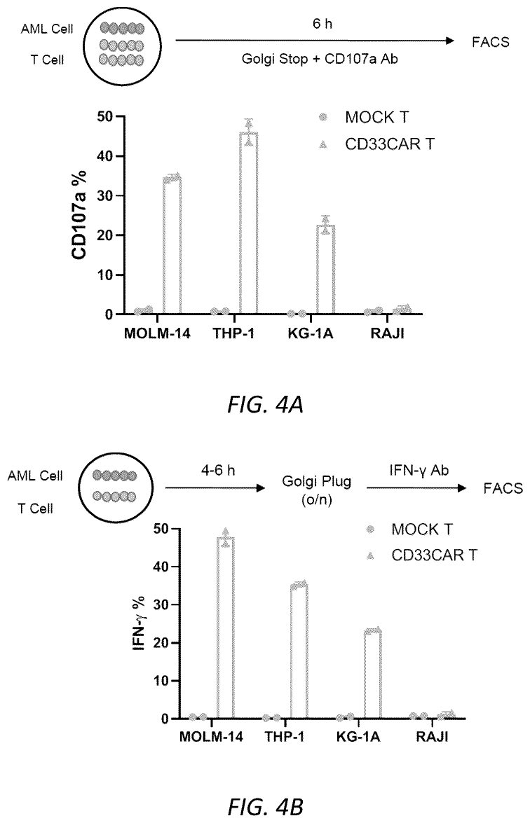

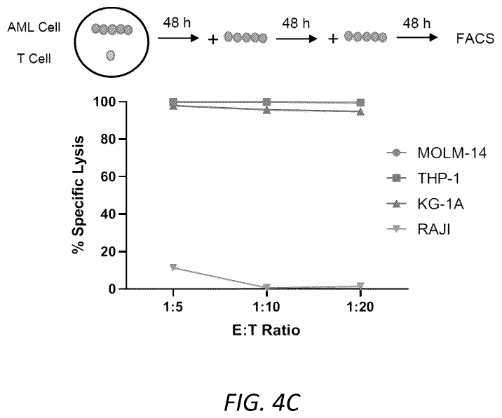

To determine if CD33 CAR T cells demonstrate selective effector functions and killing activities against CD33-positive cancer cells, the CD33 CAR T cells were grown in presence of either CD33-positive or CD33-negative cancer cells. Then CD107a degranulation levels, intracellular IFN-γ staining, and the percentage of cancerous cells killed were quantified. The schematic of the assays used is shown in FIG. 4.

For CD107a degranulation assay, mock or CD33 CAR T cells were co-cultured with target cells at an E:T ratio of 2:1 in complete X-VIVO medium supplemented with Golgi Stop and CD107a antibody for 6 hours, followed by flow cytometry. The CD33-positive AML cell lines were MOLM-14, THP-1, and KG-1A. The CD33-negative cell line used was RAJI (FIG. 4A). For intracellular IFN-γ staining assay, mock or CD33 CAR T cells were co-cultured with target cells at an E:T ratio of 1:1 in complete X-VIVO medium for 4-6 hours. The cultures were then supplemented with Golgi Plug for overnight incubation. Next, the cells were fixed, permeabilized, stained with IFN-γ antibody, and subjected to flow cytometry (FIG. 4B). For rechallenge assay, mock or CD33 CAR T cells were co-cultured with target cells at E:T ratios of 1:5, 1:10, and 1:20 for 48 hours. After the 48 hours, we added additional tumor cells to the co-culture wells to an E:T of 1:5, 1:10, or 1:20, repeated the 48 hour incubation, and added additional tumor cells to the co-culture wells to an E:T of 1:5, 1:10, or 1:20. After an additional 48 hours, flow cytometry was used to determine the % specific lysis of the tumor cells (FIG. 4C).

The CD33 (CD8) CAR T cells showed potent and specific CD107a degranulation levels, intracellular IFN-γ staining, and killing activities against CD33-positive AML cell lines, in comparison with CD33-negative RAJI cell line (FIGS. 4A-4C).

### Example 5: Validation that CD33 CAR T Cells Delivered In Vivo in a Mouse Model Exhibit Potent Anti-Tumor Activity and Confer Extended Lifespan to the Mice

To evaluate in vivo efficacy of CD33 CAR T cells to selectively target CD33-positive cells in the MOLM-14 model, CD33 CAR T cells were delivered and tumor size and survival was evaluated over time.

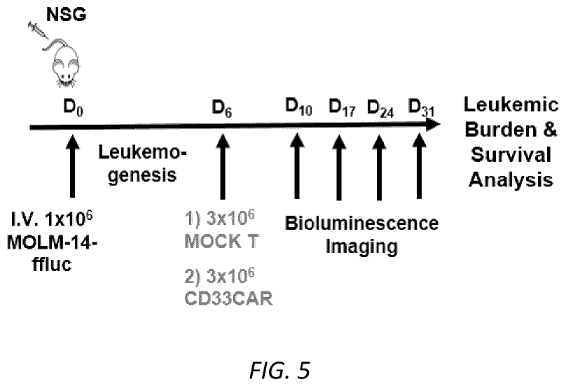

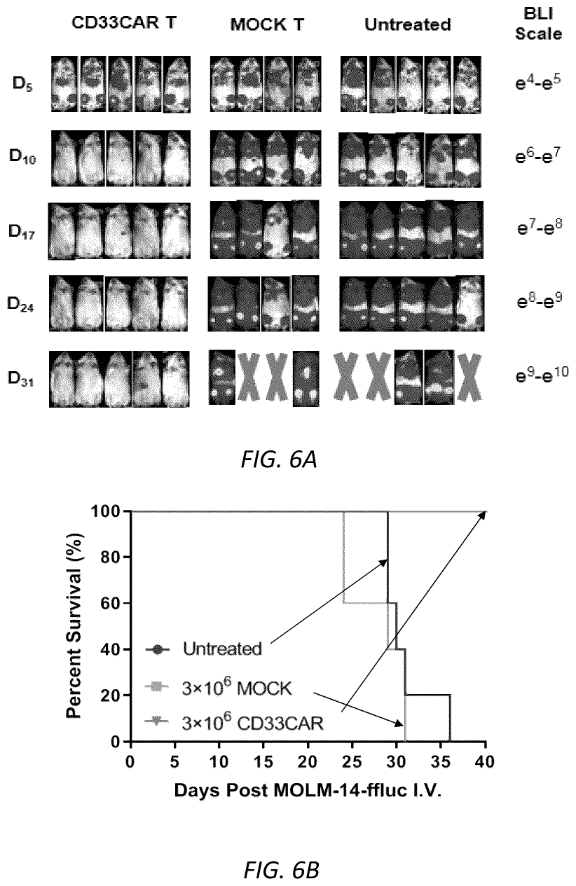

MOLM-14 cells were lentivirally transduced to express firefly luciferase (ffluc) to allow for tracking of tumor growth via non-invasive optical imaging. At 6 days post tumor intravenous injection, mice were treated with Mock or CD33 CAR T cells (3×10⁶) by systemic intravenous (i.v.) delivery (FIG. 5). Rapid anti-tumor effects were observed in mice treated with CD33 (CD8) CAR T cells via intravenous delivery, reaching a maximal anti-tumor response 1-2 weeks following treatment (FIG. 6A). Anti-tumor responses in mice were durable for 3-4 weeks. Delivery of CD33 (CD8) CAR T cells significantly extended survival of mice (FIG. 6B).

### Example 6: Decitabine Potentiates CD33 CAR T Cell-Mediated AML Killing In Vitro

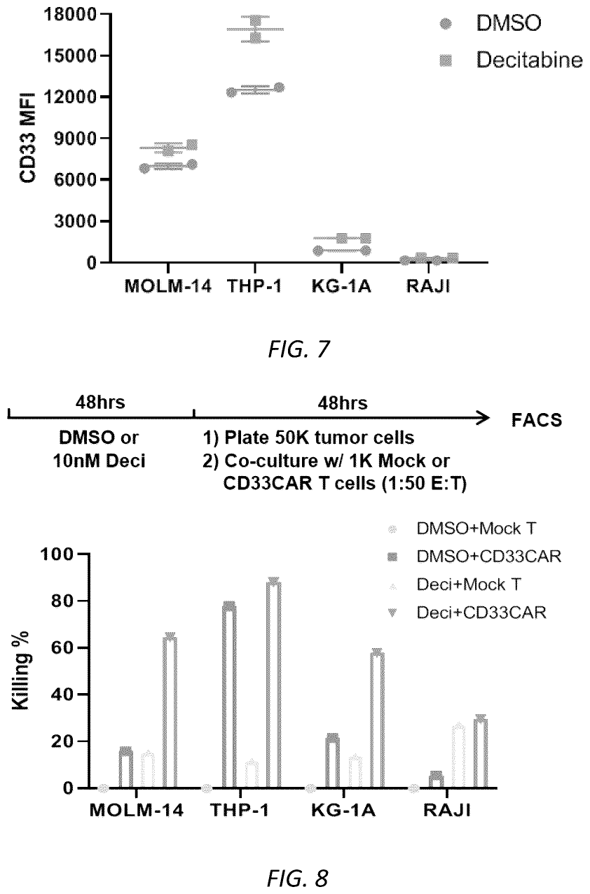

Decitabine is a commonly used hypomethylating agent (HMA) for AML treatment. We first treated AML cell lines with 100 nM decitabine or DMSO for 2 consecutive days. RAJI cell line was used as a negative control. We observed decitabine treatment led to upregulation of CD33 expression on AML cells (FIG. 7).

We hypothesized decitabine can potentiate CD33 CAR T cell-mediated AML killing. For in vitro combinational studies, AML cell lines were treated with 10 nM decitabine or DMSO for 2 consecutive days, followed by CD33 (CD8) CAR or mock T cells at an E:T ratio of 1:50. Another 48 hours later, the killing percentages (%) in each group were quantified using flow cytometry. In vitro experiments using AML cells as targets, the robust killing was seen in the group treated with decitabine followed by CD33 CAR T cells (FIG. 8).

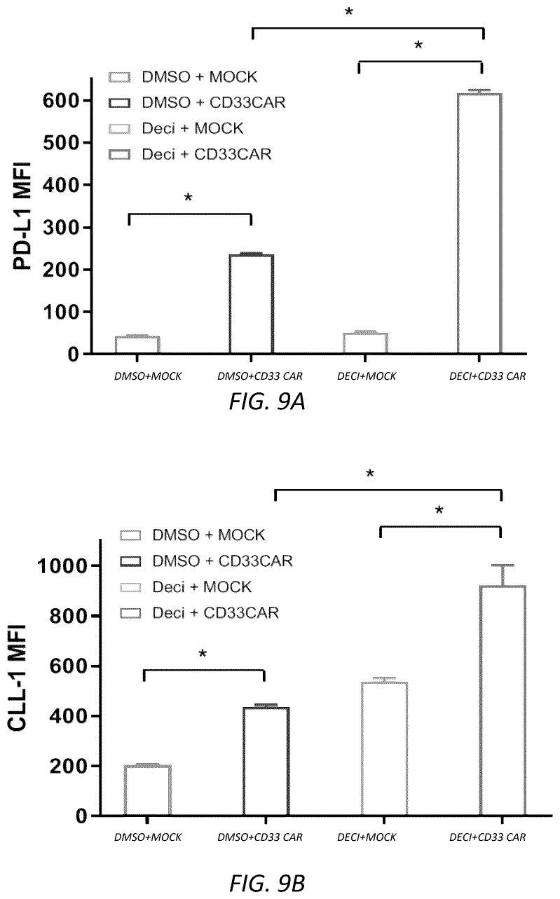

We further detected MFI of PD-L1 and CLL-1 expression on residual AML cells that survived the treatment with decitabine in combination with CD33 CAR T cells. Expression levels of PD-L1 (FIG. 9A) and CLL-1 (FIG. 9B) were significantly increased following combinational treatment in vitro.

### Example 7: Decitabine Improves the Antileukemic Efficacy of CD33 CAR T Cells In Vivo

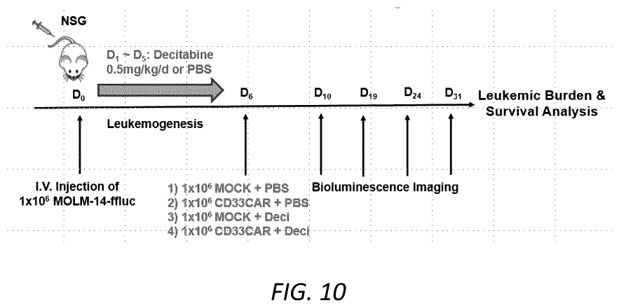

NSG mice were i.v. injected with 1×10⁶ MOLM-14-ffluc cells on Day 0. From day 1 to 5, they were administered with 0.5 mg/kg/d decitabine or PBS by intraperitoneal (i.p.) injection for 5 consecutive days. On day 6, mice were administered with 1×10⁶ CD33 (CD8) CAR (SEQ ID NO: 30) or mock T cells by i.v. injection (FIG. 10).

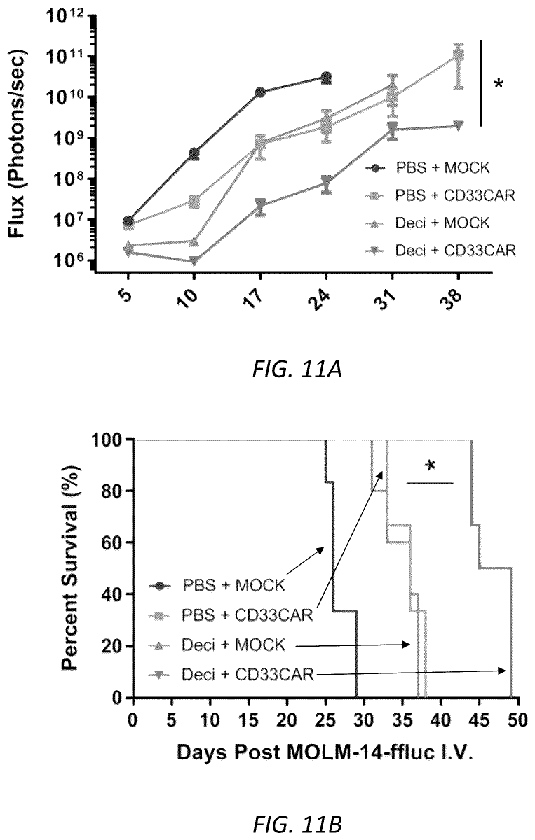

Consistent with in vitro findings, decitabine in combination with CD33 CAR T cells significantly reduced leukemic burden (FIG. 11A) and prolonged overall survival (FIG. 11B) in vivo.

### Example 8: PD-1/PD-L1 Blockade Enhances CD33 CAR T Cell Efficacy

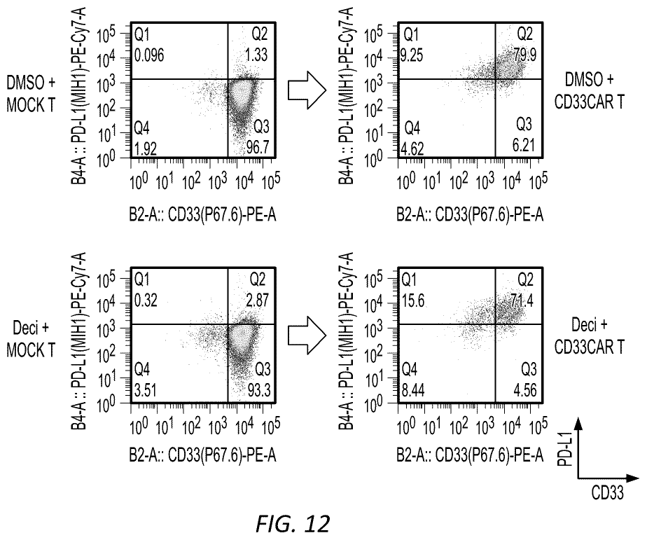

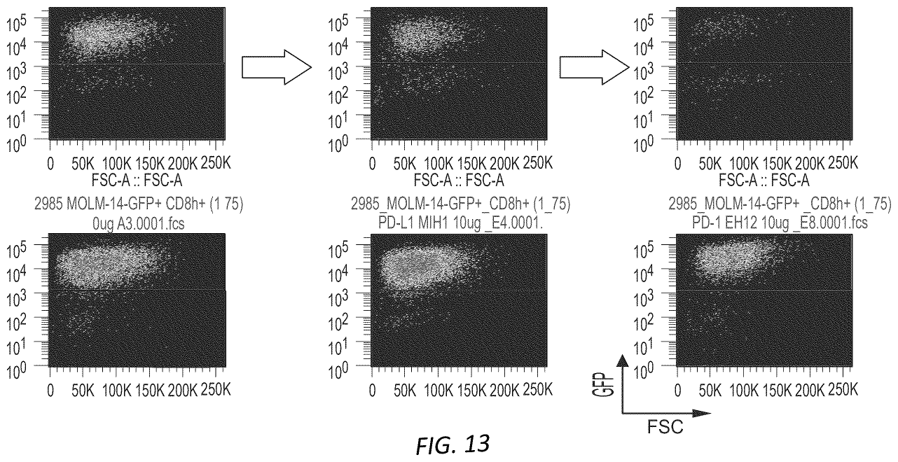

Checkpoint blockade targeting programmed death-1 (PD-1)/PD-L1 has been involved in resistance mechanisms to immune-based therapies. Residual AML cells that survived the treatment with 10 nM decitabine and 1:50 ET ratio of CD33 (CD8) CAR (SEQ ID NO: 30) T cells exhibited significantly elevated PD-L1 expression (FIG. 12). It was found that anti-PD-1 antibody (10 μg/mL) significantly enhanced the anti-AML efficacy of CD33 (CD8) CAR (SEQ ID NO: 30) T cells at an ET ratio of 1:75 (FIG. 13).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All references are herein incorporated in their entirety for any and all purposes.

What is claimed is:

1. A nucleic acid molecule encoding a polypeptide comprising a chimeric antigen receptor (CAR), wherein the CAR comprises an amino acid sequence that is at least 85%, 90%, 95%, 96%, 97%, 98%, 99%, or 100% identical SEQ ID NO: 30 or at least about 85%, 90%, 95%, 96%, 97%, 98%, 99%, or 100% identical to SEQ ID NO: 31.

2. An expression vector comprising the nucleic acid molecule of claim 1.

3. A viral vector comprising the nucleic acid molecule of claim 1,

4. A population of human T cells transduced by a vector comprising the nucleic acid molecule of claim 1.

5. The population of human T cells of claim 4, wherein the population of human T cells comprise central memory T cells, naive memory T cells, pan T cells, NK cells, or PBMC substantially depleted for CD25+ cells and CD14+ cells.

6. A method of treating a leukemia in a patient comprising administering a population of autologous or allogeneic human T cells transduced by a vector comprising the nucleic acid molecule of claim 1, wherein the leukemia comprises cells expressing CD33.

7. The method of claim 6, wherein the chimeric antigen receptor is administered locally or systemically.

8. The method of claim 6, wherein the CD33-expressing cells are cancerous T cells or T-regulatory cells.

9. The method of claim 6, wherein the chimeric antigen receptor is administered by single or repeat dosing.

10. The method of claim 6, wherein the patient is administered a hypomethylating agent prior to or in conjunction the population of autologous or allogeneic human T cells.

11. The method of claim 10, wherein the hypomethylating agent is a DNA methyltransferase inhibitor.

12. The method of claim 6, wherein the hypomethylating agent is selected from 5-azacytidine and decitabine.

13. A method of treating a cancer in a patient comprising administering immune cells comprising the nucleic acid molecule of claim 1, wherein the cancer is a hematologic cancer selected from the group acute myeloid leukemia (AML); myelodysplastic syndrome; myeloproliferative neoplasms; chronic myeloid leukemia (CML); and blastic plasmacytoid dendritic cell neoplasm.

14. A method for reducing MDSC in a patient suffering from cancer, the method comprising administering immune cells comprising the nucleic acid molecule of claim 1 to the patient.

15. The method of claim 14, wherein the patient is cancer is a solid tumor.

16. A method of preparing CD33 CAR T cells comprising:
providing a population of autologous or allogeneic human T Cells and transducing the T cells by a vector comprising the nucleic acid molecule of claim 1.
